(19) United States
(12) Patent Application Publication
(10) Pub. No.: US 2013/0072698 A1
(43) Pub. Date: Mar. 21, 2013

(54) Method and Compounds for the Preparation of Monofluoromethylated Biologically Active Organic Compounds

(71) Applicants: Emilia Perpetua Tavares Leitao, Sao Marcos (PT); William Heggie, Palmela (PT)

(72) Inventors: Emilia Perpetua Tavares Leitao, Sao Marcos (PT); William Heggie, Palmela (PT)

(73) Assignee: HOVIONE INTER LTD, Lucerne (CH)

(21) Appl. No.: 13/701,211

(22) PCT Filed: Jun. 1, 2011
(86) PCT No.: PCT/GB11/00835

§ 371 Date: Nov. 30, 2012

(30) Foreign Application Priority Data
Jun. 1, 2010 (PT) 105138

(51) Int. Cl. C07J 31/00; C07J 75/00; C07J 17/00; C07J 3/00

(52) U.S. Cl. CPC C07J 75/00; C07J 17/00; C07J 3/005
USPC 549/484; 552/610

(57) ABSTRACT

Described are processes for the preparation of monofluoromethylated organic biologically active compounds, such as Fluticasone Propionate and Fluticasone Furoate, in the presence of fluorodecarboxylating reagents such as XeF₂ and BrF₃.

### CROSS-REFERENCE TO RELATED APPLICATIONS

The present invention claims the benefit of the PCT/GB2011/000835 filed Jun. 1, 2011, which claims priority to Ser. No. PT/105138 filed Jun. 1, 2010.

### BACKGROUND OF THE INVENTION

The carbon-fluorine bond is commonly found in pharmaceutical and agrochemical products, because it is generally metabolically stable and the fluorine atom acts as a bioisostere of the hydrogen atom (Ann M. Thayer “Fabulous Fluorine” Chemical and Engineering News, Jun. 5, 2006, Volume 84, pp. 15-24). Nowadays around 20% of all pharmaceutical compounds and 30-40% of agrochemicals on the market contain fluorine. Fluorination and fluoroalkylation are the two major synthetic methods to prepare selectively fluorinated organic compounds. The monofluoromethylation (selective introduction of a CH₂F group into the organic molecule) is less studied than fluorination.

The exploration of di- and monofluoromethylated compounds as organic biologically active compounds has emerged recently. As a result, a variety of structurally diverse CH₂F-containing drugs have been developed, such as: Afloqualone, Fluticasone Propionate (Jinbo Hu; Wei Zhang; Fei wang; Chem. Commum., 2009, 7465-7478), the anaesthetic Sevoflurane and Fluticasone Furoate.

The efficient and selective incorporation of monofluoromethylated moieties into the organic molecule is beneficial for the synthesis of the target molecule. The process is usually carried out directly using CH₂FBr or indirectly, using CH₂BrI or CH₂ClI, among others. These compounds are known as hydrochlorofluorocarbons or freons (HCFCs), which is a subclass of chlorofluorocarbons (CFCs).

Every permutation of fluorine, chlorine, and hydrogen on the methane and ethane core has been examined and most have been commercialized. Furthermore, many examples containing bromine are known for higher numbers of carbon as well as related compounds. The use of this class of compounds include refrigerants, blowing agents, propellants in medicinal applications, and degreasing solvents (M. Rossberg et al. “Chlorinated Hydrocarbons” in Ullmann's Encyclopedia of Industrial Chemistry 2006, Wiley-VCH, Weinheim).

Unfortunately, due to their high stability, CFCs do not decompose in the lower atmosphere as many industrial chemicals do. In fact they are accumulating and eventually rise to the stratosphere. Ultraviolet radiation in the stratosphere breaks the CFCs apart, and the released chlorine atoms destroy the ozone layer. For this reason, the manufacture of such compounds is being phased out according to the Montreal Protocol (Pool, R. 1989. The elusive replacements for CFCs. Science 242: 666). Under the Montreal Protocol, it was agreed to start reducing their consumption and production in 2015.

The literature describes a method for replacing a carboxylic group with a fluorine group in a halogenated aliphatic carboxylic compound having the general formula, R—COOH, to prepare a fluorinated product having the general formula, R—F. The fluorodecarboxylation is carried out in the presence of XeF₂ (Timothy B. Patrick, Kamalesh K. John, David H. White, William S. Bertrand, Rodziah Mokhtar, Michael R. Kilbourn, Michael J. Welch CAN. J. CHEM. Vol. 64,1986) or BrF₃ (Patent U.S. Pat. No. 4,996,371).

### BRIEF DESCRIPTION OF THE FIGURES

FIG. 1. Schematic illustration of synthesis of fluticasone propionate and fluticasone furoate.

FIG. 2. Schematic illustration of preparation of compound of formula III-A (S-acetic acid-6α,9 α-difluoro-11 β-hydroxy, 16 α-methyl-3-oxo-17 α-(propionyloxy)androsta-1,4-diene-17β-carbothiate), wherein the R is propionate.

FIG. 3. Schematic illustration of preparation of compound of formula III-B (S-acetic acid-6α,9 α-difluoro-17 α-[(2-furanyicarbonyl)oxy]-11β-hydroxy-16 α-methyl-3-oxo-androsta-1,4-diene-17 β-carbothiate), wherein the R is furoate.

FIG. 4. Schematic illustration of preparation of compound of formula IV-B, wherein the R is furoate.

### DETAILED DESCRIPTION OF THE INVENTION

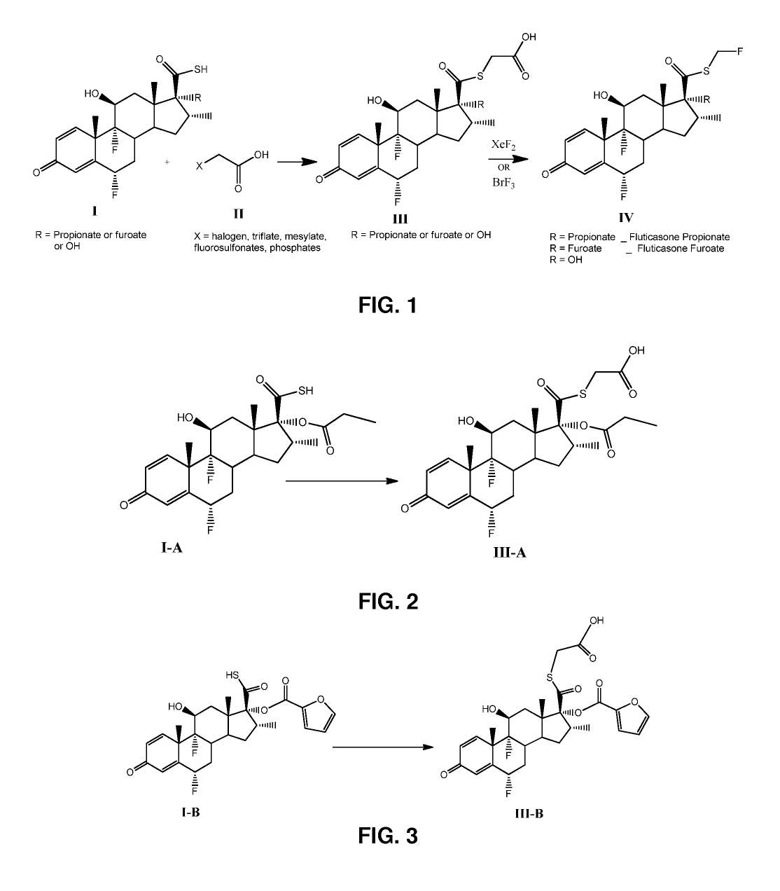

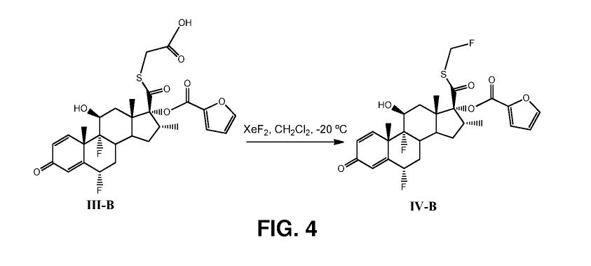

We have now discovered that, surprisingly, these reagents can be used as part of the synthesis of highly complex compounds, and can for example be applied in the synthesis of organic biologically active compounds, for example steroids, such as Fluticasone Propionate and Fluticasone Furoate as presented in FIG. 1. This avoids the use of bromofluoromethane or any other related substance that depletes the ozone layer. FIG. 1 illustrates the reaction of steroid (I) with X-acetic acid (II) to afford intermediate (III). Intermediate (III) is then fluorodecarboxylated to obtain Fluticasone Propionate or Fluticasone Furoate (IV).

Any of the methods described above (amongst others) can be used for the preparation of the organic biologically active compounds which incorporate a “CH₂F” moiety.

According to a broad aspect of the present invention, there is provided a method of preparing an organic biologically active compound containing a “CH₂F” moiety, which method comprises the steps of: reacting a compound of formula R*-SH with X-acetic acid to yield an intermediate of formula R*-S—CH₂COOH; fluorodecarboxylating the intermediate of formula R*-S—CH₂COOH with a fluorodecarboxylating reagent to yield a compound of formula R*-S—CH₂F, wherein:

R*SH is an organic multifunctional molecule;

and X is halogen, triflate, mesylate, a fluorosulfonate or a phosphate.

By “organic multifunctional molecule” it will be understood we mean to refer to any organic molecule of general formula R*SH which can serve as a precursor to the organic biologically active compound of interest and which can react with X-acetic acid according to the above scheme. Typically, the organic multifunctional molecule will be a complex molecule, and the molecule will contain at least one functional group in addition to an —SH group. Molecules having a steroidal structure (eg steroid precursors for biologically active steroid compounds) are particularly preferred. The molecule may have more than one additional functional group in addition to the —SH group.

Preferably, the molecule R*SH comprises one or more of the following functional groups: ketone, halogen, unsaturated hydrocarbon containing one or more carbon-carbon double bonds (ie an—ene group, for example, alkene), or hydroxyl. All four functional groups may be present if desired. The halogen is preferably fluorine.

Preferably, the compound of formula R*SH is a steroid molecule.

In a preferred aspect, the invention provides a method of preparing an organic biologically active compound containing a “CH2F” moiety, comprising the steps of: reacting a steroid of formula I with X-acetic acid of formula II to yield an intermediate of formula III; fluorodecarboxylating the intermediate of formula III with a fluorodecarboxylating reagent to yield compound of formula IV,

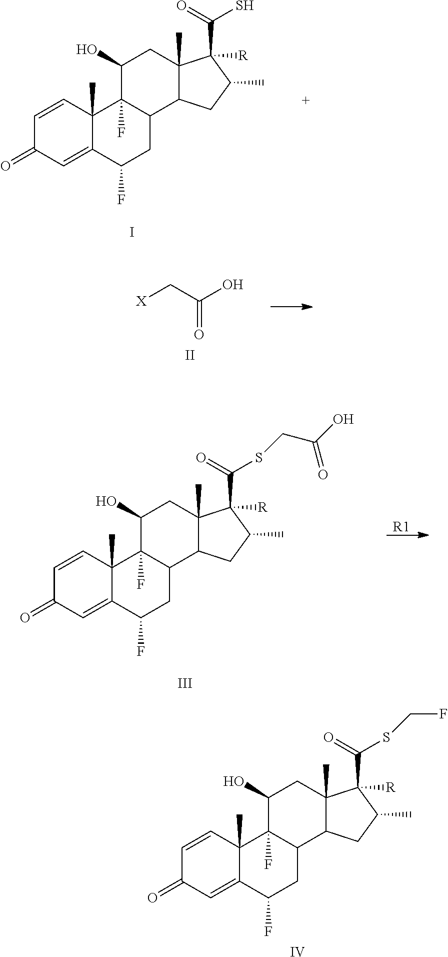

wherein:

R is propionate, furoate or hydroxyl and X is halogen, triflate, mesylate, a fluorosulfonate or a phosphate; and

R1 is a fluorodecarboxylating reagent.

Preferably, the fluorodecarboxylating reagent used in the invention is chosen from a group consisting of XeF₂ and BrF₃.

For X-acetic acid, X is preferably halogen, and preferably the halogen is bromine

In the compounds of formula I, III, and IV, R is preferably furoate or propionate.

The present invention also provides a compound of formula III,

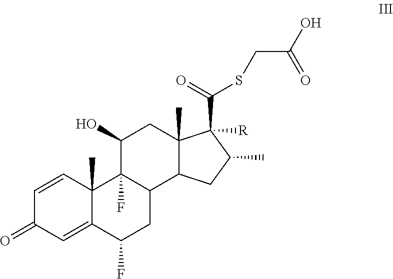

wherein R is propionate, furoate or hydroxyl.

The invention also provides the use of a compound of formula III to prepare organic biologically active compounds containing a “CH₂F” moiety. Preferably, the organic biologically active compound containing a “CH₂F” moiety is a compound of formula IV,

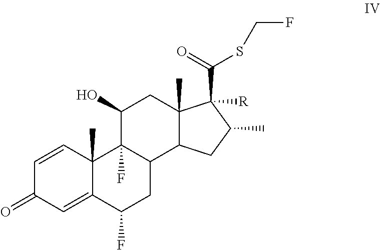

wherein R is furoate or propionate or hydroxyl.

For each of the steps in the method of the invention, the amount of reagent required (ie X-acetic acid or fluorodecarboxylating agent) per mole of substrate is suitably from about 0.9 to 7 mole equivalents. A range of about 1 to 2 mole equivalents is preferred, and is particularly suitable for the preparation of fluticasone and derivatives thereof.

Intermediate (III) can be prepared by the reaction of steroid (I) with an X-acetic acid (II) in an organic solvent and in the presence of an organic or inorganic base at a temperatures range within −70° C. and 70° C. The product can be isolated and purified by precipitation in water or water with acid or water with base, by extraction with organic solvent and/or concentration, by recrystallization in organic solvent, and/or by column chromatography. Resin and activated charcoal can also be used during the work-up to purify the products.

The product of formula IV is prepared by fluorodecarboxylation of compound III using as fluordecarboxylating reagent XeF₂ and BrF₃ and can be isolated and purified by precipitation in water or water with acid or water with base, by extraction with organic solvent and/or concentration, by recrystallization in organic solvent, and/or by column chromatography. Resin and activated charcoal can also be used during the work-up to purify the monofluoromethylated products.

### EXAMPLES

The following examples are merely illustrative and not intended to limit the scope of the invention.

### Example 1

Preparation of compound of formula III-A (S-acetic acid-6α,9 α-difluoro-11 β-hydroxy, 16 α-methyl-3-oxo-17 α-(propionyloxy)androsta-1,4-diene-17β-carbothiate), wherein the R is propionate, as shown in FIG. 2.

A solution of compound of formula I-A (1 g, 2.1 mmol), triethylamine (0.440 mL, 3.15 mmol), bromoacetic acid (0.330 g, 2.31 mmol) in dichloromethane (10 mL) was stirred at room temperature overnight. Water was added (10 mL) and the mixture extracted with dichloromethane (3×10 mL), dried with anhydrous MgSO₄, and concentrated to afford compound of formula III-A (1.451 g) as solid, as characterised further below

1H NMR (CDCl₃), 400 MHz: δ 7.22 (1H, d, J=10.1 Hz), 6.42 (1H, s), 6.37 (1H, dd, J=10.1, J=1.6 Hz), 5.39 (1H, ddd, J=48.9, J=10.3, J=6.4 Hz), 4.38 (1H, d, J=9.08 Hz), 3.75 (1H, d, J=16.0 Hz), 3.65 (1H, d, J=16.0 Hz), 3.38-3.34 (1H, m), 3.12 (2H, dd, J=14.5 Hz, J=7.2 Hz), 2.41-2.21 (5H, m), 2.02-1.98 (1H, m), 1.90-1.72 (2H, m), 1.53 (3H, s), 1.11 (3H, t, J=7.4 Hz), 1.11 (3H, s), 0.98 (3H, d, J=7.04 Hz).

13C NMR (CDCl₃), 100 MHz: δ 196.1, 185.8, 172.9, 172.6, 161.9, 161.8, 151.3, 129.9, 121.0, 120.9, 100.0, 98.2, 96.3, 86.5 (JCF=183 Hz), 71.7, 71.3, 60.4, 48.9, 48.2, 48.0, 45.7, 43.0, 36.2, 35.6, 34.1, 33.8, 33.6, 32.9, 32.8, 32.7, 32.6, 27.7, 23.0, 17.2, 16.1, 14.1, 9.1, 8.5.

FT-IR values are as follows:

FT-IR (KBr): 3407, 1743, 1670, 1631, 1608 cm⁻¹.

### Example 2

Preparation of compound of formula III-B (S-acetic acid-6α,9 α-difluoro-17 α[(2-furanyicarbonyfloxy]-11β-hydroxy-16 α-methyl-3-oxo-androsta-1,4-diene-17 β-carbothiate), wherein the R is furoate, as shown in FIG. 3.

A solution of compound of formula I-B (1 g, 1.97 mmol), triethylamine (0.410 mL, 2.96 mmol), bromoacetic acid (0.302 g, 2.17 mmol) in dichloromethane (10 mL) was stirred at room temperature overnight. Water was added (10 mL) and the mixture extracted with dichloromethane (3×10 mL), dried with anhydrous MgSO₄, and concentrated to afford compound of formula III-B (1.429 g) as solid, as characterised further below:

1H NMR (CDCl₃), 400 MHz: δ 7.56 (1H, s), 7.21 (1H, d, J=10.1 Hz), 7.09 (1H, d, J=3.4 Hz), 6.49-6.48 (1H, m), 6.42 (1H, s), 6.37 (1H, dd, J=10.1, J=1.1 Hz), 5.39 (1H, ddd, J=48.8, J=10.8, J=6.4 Hz), 4.36 (1H, d, J=9.08 Hz), 3.75 (1H, d, J=15.8 Hz), 3.67 (1H, d, J=15.8 Hz), 3.46-3.42 (1H, m), 2.48-2.26 (4H, m), 2.06-2.03 (1H, m), 1.93-1.71 (2H, m), 1.52 (3H, s), 1.17 (3H, s), 1.05 (3H, d, J=7.04 Hz).

13C NMR (CDCl₃), 100 MHz: δ 196.0, 185.7, 172.8, 161.8, 161.7, 157.0, 151.3, 147.1, 143.8, 129.9, 121.0, 120.9, 118.7, 112.0, 100.1, 98.3, 97.2, 87.5, 85.6, 71.5, 71.2, 49.3, 48.2, 48.0, 45.4, 43.2, 36.6, 35.6, 33.9, 33.8, 33.7, 32.9, 32.8, 32.7, 32.6, 23.1, 23.0, 17.2, 16.1, 8.5.

1. A method of preparing a pharmaceutically active compound containing a “CH₂F” moiety, which method comprises the steps of:
reacting a compound of formula R*-SH with X-acetic acid to yield an intermediate of formula R*-S—CH₂COOH; and
fluorodecarboxylating the intermediate of formula R*-S—CH₂COOH with a fluorodecarboxylating reagent to yield a compound of formula R*-S—CH₂F,
wherein:
R*SH is an organic multifunctional molecule;
and X is halogen, triflate, mesylate, a fluorosulfonate or a phosphate.

2. A method according to claim 1 wherein R*SH comprises one or more of the following functional groups: ketone, halogen, unsaturated hydrocarbon containing one or more carbon-carbon double bonds, or hydroxyl.

3. A method according to claim 2 wherein the halogen is fluorine.

4. A method according to claim 1, wherein the compound of formula R*SH is a steroid molecule.

5. A method of preparing a pharmaceutically active compound according to claim 1, comprising the steps of:
reacting a steroid of formula I with X-acetic acid of formula II to yield an intermediate of formula III; and
fluorodecarboxylating the intermediate of formula III with a fluorodecarboxylating reagent to yield compound of formula IV, 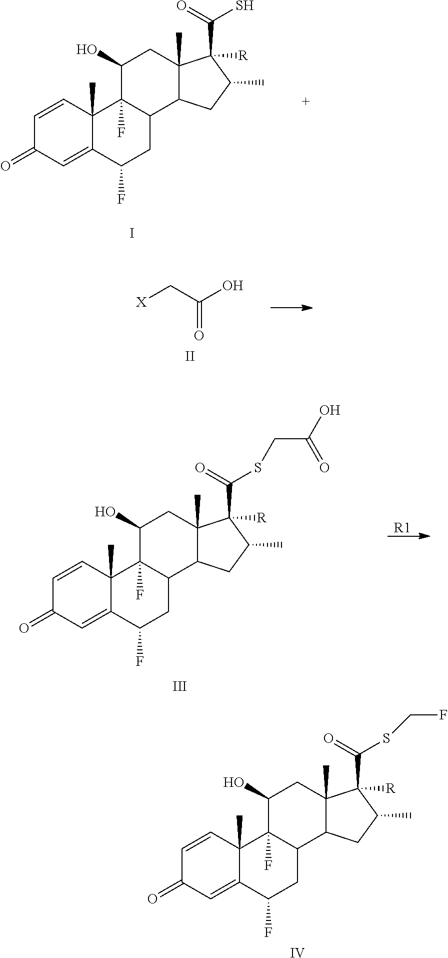
wherein:
R is propionate, furoate or hydroxyl and X is halogen, triflate, mesylate, a fluorosulfonate or a phosphate; and
R1 is a reagent.

6. A method of according to claim 1, where the fluorodecarboxylating reagent is chosen from a group consisting of XeF₂ and BrF₃.

7. A method according to claim 1, wherein X is a halogen.

8. A method according to claim 7 wherein the halogen is bromine

9. A method according to claim 5, wherein R is furoate or propionate.

10. A compound of formula III, 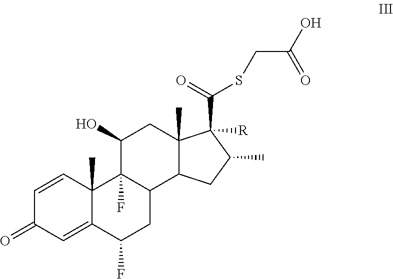
wherein R is propionate, furoate or hydroxyl.

11. A pharmaceutically active compound, the compound comprising a compound of formula III and having a formula R*S—CH₂F.

12. The compound according to claim 11, wherein the pharmaceutically active compound containing a “CH₂F” moiety is a compound of formula IV, 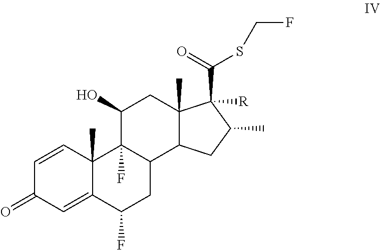
wherein R is furoate or propionate or hydroxyl.

13. A method of according to claim 5, where the fluorodecarboxylating reagent is chosen from a group consisting of XeF₂ and BrF₃.

14. A method according to claim 6, wherein R is furoate or propionate.

15. A method according to claim 7, wherein R is furoate or propionate.

16. A method according to claim 8, wherein R is furoate or propionate.
